(19) United States
(12) Patent Application Publication
(10) Pub. No.: US 2018/0344961 A1
(43) Pub. Date: Dec. 6, 2018

(54) VORTEX AIR FLOW AND NASAL CPAP

(71) Applicant: RONALD J. THOMPSON, CINCINNATI, OH (US)

(72) Inventor: RONALD J. THOMPSON, CINCINNATI, OH (US)

(21) Appl. No.: 15/995,615

(22) Filed: Jun. 1, 2018

Related U.S. Application Data
(60) Provisional application No. 62/513,786, filed on Jun. 1, 2017.

(51) Int. Cl. A61M 16/06

(52) U.S. Cl. CPC A61M 16/0666; A61M 16/0003; A61M 16/0683

(57) ABSTRACT

A respiratory interface apparatus and method for treating a patient requiring continuous positive pressure ventilation is provided. The interface can create a cylindrical stream of air, or vortex, which forms a respiratory seal with the nostrils of the patient for the transmission of pressurized air therethrough. The vortex air seal is created by annular flow nozzles which tangentially direct pressurized air in a cylindrical pattern extending into the nostrils of the patient. A separate, continuous flow of pressurized air is then transmitted through the cylindrical air seal and into the patient's airway. The air seal which is created allows the patient to receive pressurized air without having to wear a tight fitting mask. The inventive interface can include air pressure sensors and/or microprocessors for tailoring therapy to a particular patient, and can be connected to a standard CPAP machine and delivery tubing for respiratory therapy previously requiring an air-tight seal with the patient.

### CROSS-REFERENCE TO RELATED APPLICATIONS

The present invention claims the benefit of U.S. Provisional Application 62/513,786, filed Jun. 1, 2017, the disclosure of which is incorporated by reference.

### FIELD OF THE INVENTION

The present invention relates in general to devices and methods for generating and delivering continuous positive airway pressure (CPAP) therapy to patients, and in particular to a CPAP interface that uses vortex flow to create a respiratory seal with the patient.

### BACKGROUND OF THE INVENTION

Sleep apnea is a serious and potentially fatal sleep disorder if left untreated. Breathing repeatedly slows and intermittently stops (apnea) for periods of time while a person is sleeping. This erratic breathing pattern can cause a dangerous drop in the arterial oxygen supply to the brain and heart muscles, increasing the chances of hypertension, stroke, heart attack, and sudden death. The most common form of sleep apnea is obstructive sleep apnea, which occurs when upper airway muscles relax and the weight of the surrounding tissues narrows or closes the upper airway. Oxygen levels drop, and the body sends out hormonal signals in response, often causing the person to repeatedly wake up sweating and agitated throughout the night.

Continuous positive airway pressure (CPAP) therapy typically entails the continuous transmission of positive pressure into the lungs of a spontaneously breathing patient throughout the respiratory cycle. Technological advancements have led to moderate success for CPAP machines as a non-surgical treatment for obstructive sleep apnea, as well as other respiratory insufficiencies such as excessive snoring, sinusitis, hay fever, and allergic rhinitis. Indeed, the global sleep apnea device market has been projected to grow from about $4.6 billion in 2016 to $6.7 billion by 2021 (USD), with positive airway pressure devices accounting for the largest share of this market. However, while airway pressure support systems such as CPAP are now common in both hospital and home care settings, most existing devices are not easy to use, and patient compliance can be difficult.

Conventional CPAP machines generally include a source of pressurized air, supply tubing connecting the air source to the patient, and patient/tubing respiratory interfaces of various types including oral, nasal and tracheal. For home therapy and most hospital settings, the respiratory interface is typically a mask, such as a full-face mask, nasal mask, or nasal pillow. Masks are typically strapped to the face of a wearer by headgear to provide air-tight sealing against the mouth and/or nostrils. Indeed, for most CPAP systems to be beneficial the mask must be substantially leak-proof and/or air-tight, because leaks will dissipate the supplied pressure into the atmosphere and decrease effectiveness.

In addition to the challenges of keeping an air-tight seal, patients frequently struggle at night because such masks can be uncomfortable, wobbly, and heavy. These challenges, if not addressed properly, can compromise the patient's compliance with the prescribed therapy. Nasal masks have an advantage in that the point of contact with the nares/nostrils, i.e. the area of the seal, is much reduced; however, they still require an air-tight seal with the nostrils and require a headband or harness to maintain the pressure, resulting in the same patient discomfort noted with face masks. Long-term compliance with conventional CPAP systems is therefore a problem. The tight fit necessary for an air-tight seal can make it difficult to sleep through the night, and the system often goes unused.

Efforts have been made to improve current CPAP systems. For example, U.S. Pub. 2015/0267695 to Marsh envisions a self-contained maskless, tubeless nasal CPAP system having a plurality of piezoelectric micro-blowers which pump air through nostril-sealing prongs. While such a device would be useful, to date a working product has not been made available for use, and the air-tight seal required, as noted above, can be uncomfortable during sleep.

U.S. Pat. No. 9,132,250 to Allum et al. teaches a non-invasive open-airway ventilation (NIOV) system which includes a nasal mask interface which need not completely seal the patient's nostrils. The nasal mask includes internal jet nozzles in a Venturi arrangement with the source of pressurized air, which creates an area of positive pressure laminar flow within the mask. While useful as therapy for active patients compared to standard oxygen therapy, leakage and loss of the delivered air pressure may occur, and some patients may require more pressurized air than can be delivered.

U.S. Pat. No. 7,798,148 to Doshi et al. teaches disposable nasal expiratory positive airway pressure (nasal EPAP) inserts including one-way valves which are sealed over or within the nostrils, typically via an adhesive tape. The small inserts utilize the patient's own breathing to create a positive end-expiratory pressure with minimal inspiratory resistance. While these devices can be useful for travel or in places lacking electricity, the air-tight inserts can be uncomfortable, and pressurized air provided by an external source is still needed for many patients.

In light of the above it is apparent that despite recent advancements there remains a need in the art for an improved means for delivering pressurized air. For example, it would useful be to provide an interface for a CPAP device that does not require air-tight sealing against the mouth and/or nostrils. It would also be advantageous to provide a respiratory interface for use in either a hospital or home care setting system that is light-weight, comfortable and easy to use. It would also be useful to provide a respiratory interface which results in better long-term patient compliance with sleep apnea therapy and improved respiratory and cardiovascular health.

### SUMMARY OF THE INVENTION

The present invention is an improved apparatus and method for interfacing/connecting a patient with a positive pressure source. The invention employs the creation of a vortex flow pattern around the delivered pressurized air to create a seal between the patient and the pressurized air.

A first aspect of the invention relates to a respiratory interface for a ventilation system, the interface comprising: (a) a body portion defining a main fluid flow path, the main fluid flow path having openings at opposing ends of the body portion for connection to supply tubing from a pressurized air source; and (b) a pair of hollow nasal cylinders extending from the body portion for placement adjacent to the user's nostrils, each nasal cylinder including an air passage fluidly connected to the body portion for transmission of pressurized air into the nostril, each nasal cylinder further including an annular fluid flow nozzle.

A second aspect of the invention relates to a respiratory interface for a ventilation system, the interface comprising: (a) a body portion defining a fluid flow path, the body portion having openings at opposing ends for connection to supply tubing from a pressurized air source; and (b) a pair of hollow nasal cylinders extending from the body portion for placement adjacent to the user's nostrils, each nasal cylinder including: (i) an air passage fluidly connected to the fluid flow path of the body portion for transmission of pressurized air into the nostril; and (ii) an internal annular cavity, each annular cavity having an opening at the end of the body portion for connection to the supply tubing from the pressurized air source and defining an annular fluid flow nozzle for directing pressurized air in an axial direction around the main air passage to provide a constant air seal around the main air passage while inhibiting leakage of pressurized air delivered through the fluid flow path into the nostril.

A third aspect of the invention relates to a method for providing pressurized air to a patient's airways, the method comprising the steps of: (a) providing a respiratory interface, the interface comprising: (i) a body portion defining a main fluid flow path, the main fluid flow path having openings at opposing ends of the body portion for connection to supply tubing from a pressurized air source; and (ii) a pair of hollow nasal cylinders extending from the body portion for placement adjacent to the user's nostrils, each nasal cylinder including an air passage fluidly connected to the body portion for transmission of pressurized air into the nostril, each nasal cylinder further including an annular fluid flow nozzle for forming a cylindrical stream of air; (b) connecting the body portion and the nasal cylinders to the pressurized air source; and (c) operating the pressurized air source to provide positive pressure air flow through the main fluid flow path, the air passage of each nasal cylinder and the annular fluid flow nozzle of each nasal cylinder, wherein the air flow through the annular fluid flow nozzles forms a cylindrical stream of air which creates a respiratory seal between the patient and the supply tubing, and wherein pressurized air is transmitted through the main fluid flow path and the air passages to the patient's airways

While the nature and advantages of the present invention will be more fully appreciated from the following drawings and detailed description, showing the contemplated novel construction, combinations and elements as herein described, and more particularly defined by the appended claims, it is understood that changes in the precise embodiments of the present invention are meant to be included within the scope of the claims, except insofar as they may be precluded by the prior art.

### BRIEF DESCRIPTION OF THE DRAWINGS

FIG. 1 is a schematic cross-sectional view of one embodiment of a respiratory interface according to the invention;

FIG. 2 is a schematic top view of the embodiment of FIG. 1;

FIG. 3 is a schematic cross-sectional view of the embodiment of FIG. 1 showing the device inserted into a patient's nose.

### DETAILED DESCRIPTION OF THE INVENTION

The present invention provides an apparatus and method for treating a patient requiring positive pressure ventilation. The apparatus can create a cylindrical respiratory seal with the nostrils of the patient for the transmission of pressurized air therethrough, without any physical seal required between the patient and the apparatus. An annular, vortex air seal is created by tangentially directed, pressurized air and extends into the nostrils of the patient, allowing a separate, continuous flow of pressurized air to pass through the air seal and into the patient's airway.

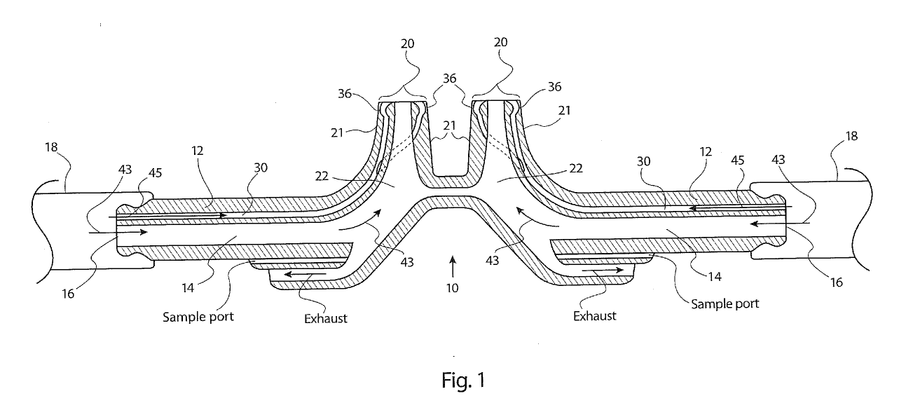

Referring now to the drawings in detail, FIG. 1 is a schematic cross-sectional view of a respiratory interface 10 for a ventilation system according to one embodiment, which illustrates the system during pressurized air delivery, typically during spontaneous inspiration. The interface 10 includes a body portion 12 defining a main fluid flow path 14. Air supply openings 16 at opposing ends of the flow path 14 defined by the body portion can be configured to connect to supply tubing 18 from a pressurized air source such as a CPAP machine (not shown). A pair of hollow nasal cylinders 20 are designed for non-sealing placement adjacent to the user's nostrils. For the purposes of this invention, the phrase “adjacent to” means bordering the user's nostrils, just beneath the user's nostrils, or just within the user's nostrils.

Each nasal cylinder 20 is generally made up of a cylindrical wall 21, continuous with and extending from the body portion 12. Each cylindrical wall 21 includes an air passage 22 fluidly connected to the main fluid flow path 14 of the body portion for transmission therethrough of pressurized air into the patient's nostril. In contrast to nasal masks or pillows, the nasal cylinders 20 do not require an air-tight physical connection or physical seal with the nostrils. The wall 21 of each nasal cylinder 20 includes an internal annular cavity 30 which begins at the same or ipsilateral side of the interface 10 as the respective nasal cylinder 20. Each annular cavity 30 is separate from but typically begins near the air supply opening 16 of the ipsilateral fluid flow path 14. As can be appreciated from viewing FIG. 1, the annular cavities 30 are separate from the main fluid flow path 14 of the body portion 12 and the air passages 22 of the nasal cylinder 20, and can be fluidly connectable either to the supply tubing 18 from the pressurized air source as shown in FIG. 1, or to a separate source of pressurized air (not shown). In any event, each annular cavity 30 passes between the inner and outer surfaces of the nasal cylinder wall 21, and defines an annular fluid flow nozzle 36 which transmits the air flow in an axial direction.

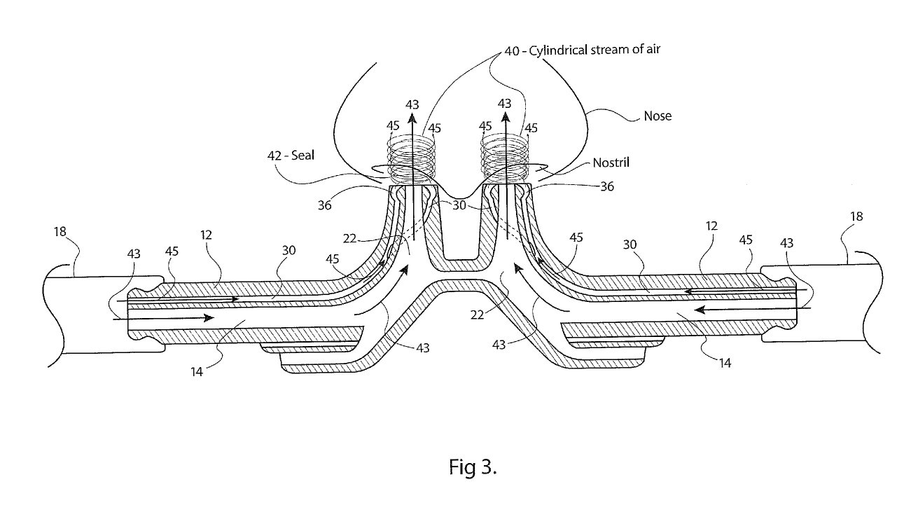

Each annular fluid flow nozzle 36 exits the distal end 23 of the wall 21 of its nasal cylinder 20, such that pressurized air passing through the flow nozzle creates a virtual extension of the cylinder wall 21. In use, the passage of air through the annular cavity 30 and flow nozzle 36 causes the air to form a cylindrical or cone-shaped stream 40 (see FIG. 3) encircling and extending from the distal ends of each nasal cylinder. This stream 40 can create a respiratory seal with the internal diameter of the nostril of the patient. An exhaust flow path may be included as shown in FIG. 1, which may permit the patient to exhale through the interface device 10 in addition to inspiring through it. The interface 10 may also include a sample port as labeled in FIG. 1 for sampling gas content within the interface 10. Such a port may also be used for additional functions, for example, a pressure sensing port, gas sampling or as a humidification delivery lumen.

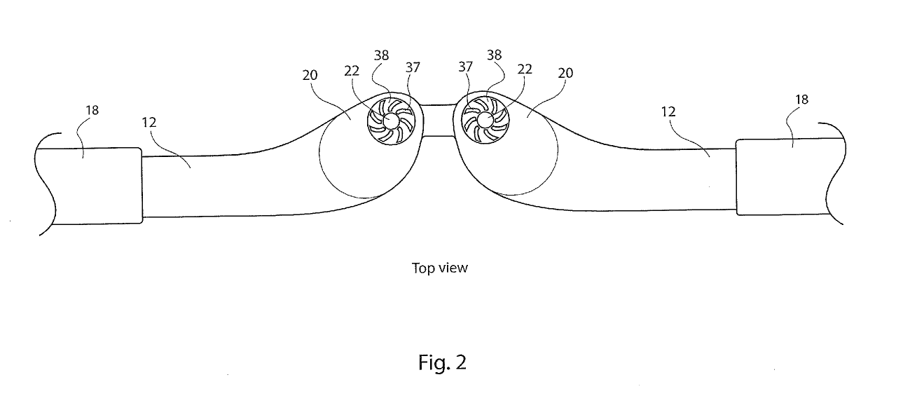

FIG. 2 shows that each of the annular fluid flow nozzles 36 preferably include an array of radially outwardly extending fluid directing spacers, vanes or jets 38, which are configured to create a vortex with the pressurized air flowing through the annular cavities 30, thereby discharging the air from the nozzles 36 in the aforementioned cylindrical or cone-shaped stream 40, forming an annular wall of air. This annular wall or cylindrical stream of air 40, as illustrated in FIG. 3, is created as pressurized air passes through the array of arcuate openings 37 between the jets or vanes 38. With continuous pressurized air flow, the cylindrical stream of air 40 produced by the vortex can extend distally from the nasal cylinder 20 and create a respiratory seal 42 with the patient, for example, via the internal diameter of the nostril. This cylindrically shaped respiratory seal 42, which is essentially an air seal connecting the pressurized air flowing through cavities 30 with the user's respiratory system, allows the pressurized air flowing through the main fluid flow path 14 of the body portion 12 to pass through the air passages 22 of the nasal cylinders 20, through the cylindrical stream 40 extension created by the annular cavities 30, and to directly enter the patient's airway via the nostrils, without any substantial leakage or loss of pressure.

Looking at FIG. 3, a first flow of pressurized air, depicted by arrows 43, is transmitted from supply tubing 18 through the air supply openings 16, through the main fluid flow path 14 and delivered to the patient through the nasal cylinder air passages 22. A second flow of pressurized air is depicted by arrows 45, which is transmitted through the annular cavities 30 and the annular flow nozzles 36 to create the cylindrical wall of air 40 and the annular seal 42 discussed above. Thus, leakage of airflow 43 is prevented and pressure is maintained by airflow 45. In one embodiment, at least one pressure sensing port may be included, for example port 44, to give the respiratory interface 10 the ability to determine flow rates and volumes flowing through the interface during inhalation and exhalation. Optionally, multiple pressure sensing port locations (not shown) can be used to measure spontaneous breathing pressures, or the pressure of the air being delivered.

The present invention employs a vortex element to create a seal at the patient interface in combination with continuous delivery of pressurized air to the respiratory system. The invention applies a vortex flow similar to that used in certain surgical insufflation systems, disclosed for example in U.S. Pat. No. 7,182,752 to Stubbs et al., U.S. Pat. No. 8,798,223 to Steams et al., and marketed as the AirSeal® trocar and cannula line of products. The AirSeal® maintains a pneumoperitoneum without a mechanical seal by creating a vortex with pressurized air through a high pressure nozzle at a tangential attitude. The AirSeal® includes a supply line which provides constant pressurized air to vortex-producing, tangential high pressure nozzles in the proximal trocar.

With the above concept applied to the a nasal CPAP system, the present invention can provide a suitable air-tight seal at the patient interface without requiring the patient to wear a tight fitting, leak proof mask. The vortex air flow provided by the annular flow nozzles of the present invention can create a seal at the patient interface, through which continuous positive pressure and airflow can be passed. The inventive device can typically be used for treating upper airway obstructions and collapse, all proximal to the hard trachea, but it is envisioned that the inventive device could also distend the lower pulmonary tree and alveoli. The inventive sealing device can also include air pressure sensors and/or microprocessors for tailoring therapy to a particular patient. The inventive concept includes using a standard CPAP machine and delivery tubing, with or without O₂. However, the invention is believed to be adaptable to any therapy previously requiring an air-tight seal with the patient. Further, it is envisioned that the inventive interface apparatus can be manufactured to be either disposable or reusable after being sterilized.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative system and method, and illustrated examples shown and described. Accordingly, departures may be made from such details without departing from the scope of the invention.

What is claimed is:

1. A respiratory interface for a ventilation system, the interface comprising:
a) a body portion defining a main fluid flow path for connection to supply tubing from a pressurized air source; and
b) a pair of hollow nasal cylinders extending from the body portion for placement adjacent to the user's nostrils, each nasal cylinder including an air passage fluidly connected to the body portion for transmission of pressurized air into the nostril, each nasal cylinder further including an annular fluid flow nozzle for forming a cylindrical stream of air.

2. A method for providing pressurized air to a patient's airways, the method comprising the steps of:
a) providing a respiratory interface, the interface comprising:
i) a body portion defining a main fluid flow path for connection to supply tubing from a pressurized air source; and
ii) a pair of hollow nasal cylinders extending from the body portion for placement adjacent to the user's nostrils, each nasal cylinder including an air passage fluidly connected to the body portion for transmission of pressurized air into the nostril, each nasal cylinder further including an annular fluid flow nozzle for forming a cylindrical stream of air;
b) connecting the body portion and the nasal cylinders to the pressurized air source; and
c) operating the pressurized air source to provide positive pressure air flow through the main fluid flow path, the air passage of each nasal cylinder and the annular fluid flow nozzle of each nasal cylinder, wherein the air flow through the annular fluid flow nozzles forms a cylindrical stream of air which creates a respiratory seal between the patient and the supply tubing, and wherein pressurized air is transmitted through the main fluid flow path and the air passages to the patient's airways.
